(19) United States
(12) Patent Application Publication
(10) Pub. No.: US 2010/0036332 A1
(43) Pub. Date: Feb. 11, 2010

(54) ASPIRATION DRAINAGE SYSTEM COMPRISING A MATERIAL DEPOT WITH AN ANTI-BACTERIAL ACTION

(76) Inventor: Dietmar Wolter, Hoisdorf (DE)

Correspondence Address: MORRISON & FOERSTER LLP, 1650 TYSONS BOULEVARD, SUITE 400, MCLEAN, VA, 22102, US

(21) Appl. No.: 12/520,786

(22) PCT Filed: Dec. 12, 2007
(86) PCT No.: PCT/EP07/10897

§ 371 Date: Oct. 26, 2009

(30) Foreign Application Priority Data
Dec. 20, 2006 (DE) 102006060934.4

(51) Int. Cl. A61M 1/00; A61M 39/02

(52) U.S. Cl. 604/265

(57) ABSTRACT

The subject matter of the invention is an aspiration drainage system for aspirating secretions and other fluids out of the human or animal body using a suction tube and at least one material depot, arranged at the end of the suction tube to be inserted into the body, for dispensing an antibacterial active ingredient material. The surface of the material depot is larger than the envelope of the material depot. The increased surface of the material depot effects a higher level of the antibacterial active ingredient material and permits effective destruction in situ of both sensitive pathological germs and partially resistant germs.

### REFERENCE TO RELATED APPLICATIONS

This application is a national stage application under 35 USC 371 of International Application No. PCT/EP2007/010897, filed Dec. 12, 2007, which claims the priority of German Patent Application No. 10 2006 060 934.4, filed Dec. 20, 2006, the contents of which prior applications are incorporated herein by reference.

### FIELD OF THE INVENTION

The invention relates to an aspiration drainage system for aspirating secretions and other fluids out of the human or animal body, which is provided with a material depot with an antibacterial action.

### BACKGROUND OF THE INVENTION

It is known that the application of aspiration drainage systems, e.g. Rendon's aspiration drainage systems, can significantly reduce the formation of hematomas and accumulation of lymph in surgical wounds. This leads to improved wound healing because the tissue layers can lie against one another and as a result heal directly.

Every surgical intervention leads to contamination of the wound with germs. In an aspiration drainage system, which is led to the outside via an additional opening in the skin outside of the actual wound, it can be observed that there is also an invasion of germs from the outside via the drainage, the lumen or the outer side itself.

This process of germ invasion during drainage thus occurs not only on the outer side of the drainage, but also in the case of improper use, in particular when individual system components are exchanged via the drainage lumen. If the treating medical practitioner or the carer does not, for example, wear sterile gloves and does not effect a sufficient disinfection, it is possible for pathological germs, which populate the entire system and the wound, to contaminate the drainage lumen and its contents during the exchange of individual system components, for example when exchanging the vacuum flask.

EP 0 476 503 discloses the fact that it is possible to counteract the germs by using a material depot with an antibacterial action, for example a poly(methyl methacrylate) (PMMA) plug loaded with gentamycin at the end of the suction tube of the aspiration drainage system which is to be inserted into the body. The antibacterial material, dispensed locally from the material depot, does not only act against germs which entered the wound through the outer side of the drainage or through the lumen, but also against germs that entered the wound during the operation.

The material with an antibacterial action leaves the material depot in the wound and reaches the wound together with the secretions and other fluids, and is admixed there with the wound fluid. At the same time, the wound secretion reaches the openings of the suction tube by means of which it is aspired. Compared to a systemic antibiotics therapy, this makes it possible to obtain higher active ingredient concentrations at the location of the wound without the overall body being loaded with curative amounts of antibiotics.

It is known that the destruction of the germs is not only dependent on the presence of the antibiotic in the environment surrounding the bacterium, but also on the concentration of the antibiotic (antibiotics level) in the tissue. The local treatment of the wound with an aspiration drainage system with a material depot with an antibacterial action is often still effective when microbiological tests verify a “simple” resistance to antibiotics, since bacteriological testing usually only examines such concentrations of antibiotics which can be attained during the course of an intravenous therapy.

It is known that, even when using aspiration drainage systems with a material depot with an antibacterial action of the type mentioned initially, the case can occur where not enough of the antibiotic is dispensed. The antibacterial material (antibiotic) dispensed by the material depot reaches the openings of the suction tube, by means of which it is aspired, too quickly in the case of relatively strong bleeding, and hence relatively high aspiration amounts, before the desired antibiotics level has been reached in the wound.

### SUMMARY OF THE INVENTION

The object of the invention is to provide an aspiration drainage system of the type mentioned initially which does not have the abovementioned disadvantages.

In accordance with claim 1, the surface of the material depot, arranged at the end of the suction tube of the aspiration drainage system to be inserted into the body and via which the antibacterial active ingredient material is dispensed into the human or animal body, is larger than the envelope of the material depot.

The envelope describes a closed surface spanned around a body. In a cross-sectional view, it holds true that the envelope touches each curve of a family of curves at one point. By way of example, the envelope of a sphere with a base radius r and channels extending into the interior space of the sphere is determined by the surface of the sphere, as is found to result from the base radius (4πr²).

The invention has realized that the amount of antibacterial active ingredient material dispensed via the surface of the material depot depends on the content of the active ingredient material in the carrier or depends on the loading of the carrier material with the active ingredient material, and that this content or load by the carrier material is subject to a predefined upper limit. The invention has likewise realized that the antibiotic from the carrier material is only released from the uppermost layer of the carrier material. During examinations of explanted gentamycin-PMMA chains, also known as Septopal® chains to a person skilled in the art, and also of explanted gentamycin-containing bone cement material when prostheses were replaced, it could be verified that the antibiotic material gentamycin is only released from the outer region, which is a few millimeters thick, while the deeper layers have unchanging proportions of antibiotics. Moreover, the invention has realized that increasing the surface of the material depot makes it possible to release more antibiotic per unit time, that is to say a sufficiently high level of antibiotics can be set, even if there is a rapid removal of the antibiotic, with the wound secretion and other fluids, through the openings of the suction tube. This makes it possible to counteract under-dosage of the antibiotics. The high local concentration of antibiotics leads to an effective destruction of not only sensitive pathological germs, but also of partially resistant germs.

The invention has likewise realized that the material depot according to the invention does not lead to an overdose of the antibiotics, which is also considered to be problematic. It was found that the antibiotic dispensed from the material depot to the wound fluid is distributed in the wound fluid by the dynamics of the wound fluid, for example by a change in the position of the patient or by muscular activity, and an antibiotics level which is harmless to the patient is set locally, and also in the blood level in general. To the extent that an overdose cannot be excluded in an individual case, or if a delayed release of the antibiotic is desired, it is expedient to accordingly set the speed of the quantity of antibiotics dispensed. This can be effected by suitable proportions of the antibiotic in the material depot and/or of additives contained in the material depot which delay the release of the antibiotic. The speed of release can also be controlled by the material depot being compressed by different strengths or, for example, having a foam-like design.

The surface of the material depot can be increased by means of the most diverse surface structures, for example by depressions, hollow recesses, indentations and protuberances such as channels, folds, slits, lamellae, combs and bores. The channels, folds and slits are established along, across or, in a thread-like manner, in the surface of the material depot, preferably along the aspiration drainage tube axis or in a thread-like manner about the aspiration drainage tube axis. Advantageously, the surface structures do not damage, irritate or negatively affect the tissue in situ. Thus, the material depot advantageously does not have sharp edges so that damage to the tissue, the nerves and the vessels, and secondary hemorrhage can be avoided to the greatest possible extent during the removal of the aspiration drainage system. The arrangement according to the invention of channels, folds and slits along the aspiration drainage tube axis supports the unimpeded slipping of the aspiration drainage system when being pulled out of the wound.

The depths or heights of the surface structures are expediently selected such that, on the one hand, the stiffness of the carrier material is not negatively affected and, for example, an undesired breaking-off of carrier material is avoided during the insertion or removal of the suction tube with the terminally arranged material depot; on the other hand, the material thickness between the adjacent structures of the material depot is so thick that an active ingredient, which is as effective as possible, is dispensed from the carrier material in the wound. Thus, by way of example, lamellae, that is to say long longitudinal grooves, have a very large surface and stability measured in terms of the material expenditure, in particular when they are arranged in groups with their surfaces parallel or approximately parallel to each other and are connected to one another. If it is taken into account that the antibiotic material gentamycin is only released from the carrier material PMMA in the wound from an enveloping region which is a few millimeters thick, the material thickness between the adjacent structures of the material depot should preferably only be a few millimeters, preferably at most 5 mm, more preferably 3 mm, particularly preferably 1 to 3 mm.

The structuring of the material depot according to the invention permits the increase of the surface of the material depot compared to the envelope by at least a factor of 1.1, 1.2, 1.3, 1.4, 1.5, 1.6, 1.7, 1.8, 1.9 or by a multiplicity of times (by at least a factor of 2, 3, 4, 5, 6, 7, 8, 9, 10, 11-100, 100-200, 200-300, 300-400, 400-500, 500-600, 600-700, 700-800, 800-900 or 900-1000).

Expediently, the material with an antibacterial action is an antibiotic, gentamycin being particularly expedient. As an alternative to, or in combination with, gentamycin, it is also possible to use further antibiotics, such as vanvomycin and/or clindamycin.

The material with an antibacterial action can be enclosed in a reabsorbable carrier material (e.g. polyglycolide-lactide or collagen) or a non-reabsorbable material (e.g. PMMA). Preferably, the material depot has, at least in parts, open pores on its surface. The open pores permit simplified dispensing of the antibacterial active ingredient material in the wound. In accordance with a preferred embodiment of the invention, the reabsorbable material depot is arranged detachably at the end of the suction tube to be inserted into the body, for example by means of a predetermined breaking point between the suction tube and the material depot. When the aspiration is complete, the material depot can still be destroying germs.

The basic geometric shape of the material depot, formed by the envelope, is preferably a shape selected from the group composed of cylinder, sphere, cone and the combination of these basic shapes such as the teardrop shape. In principle, every basic shape is preferred which permits, gently for the patient, the insertion of the end of the suction tube with the material depot into the body and/or the removal thereof. “Gently” means in particular that the wound opening for inserting and/or removing the suction tube with the material depot can be kept as small as possible, but nevertheless permits simple removal.

The aspiration drainage system according to the invention can furthermore have a chain of material depots of the type according to the invention at the end of the suction tube to be inserted into the body. In other words, the material depot arranged at the end of the suction tube to be inserted into the body is lengthened by at least one further material depot in the manner of a chain element. The chain elements can be arranged on a central wire. The chain elements can, for example, have a spherical basic shape, analogous to the Septopal® chains known from the prior art, or the shape of a roller, for example in the shape of a solid cylinder. The adjacent chain elements can also have surfaces which correspond to each other. The surfaces can, for example, be of convex-concave design. This affords the possibility of an articulated movement of the chain elements which makes it easier to insert the aspiration drainage system into the body, (re)position it if required or remove it, even in the case of a small distance between the adjacent chain elements. Such forms of chains have the advantage that they are flexible and can be matched to the local conditions.

Here, reference is made again to the fact that the abovementioned advantages of the aspiration drainage systems are not only afforded in the case of aspiration drainage systems inserted into surgical wounds. Rather, there are similar advantages in different cases in which a secretion has to be aspired from the human or animal body. The thorax drainage and a urinary catheter are mentioned here as examples thereof.

### BRIEF DESCRIPTION OF THE DRAWINGS

In the following text, the invention will be described on the basis of advantageous embodiments with reference to the attached drawings, in which:

FIG. 1 shows a first embodiment of a suction tube with a material depot which can be used for the aspiration drainage system according to the invention;

FIG. 2 shows a second embodiment;

FIG. 3 shows a third embodiment; and

FIG. 4 shows a fourth embodiment.

### DESCRIPTION OF THE INVENTION

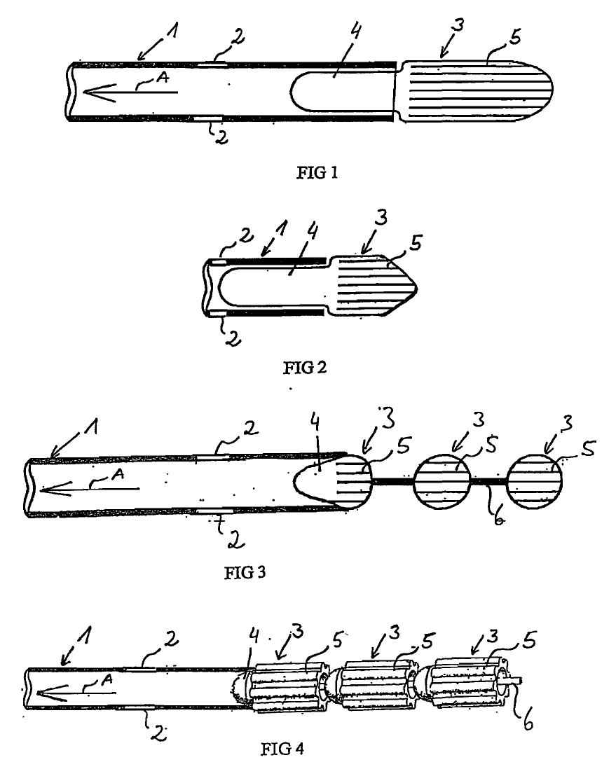

FIG. 1 shows a cross section of a suction tube 1 which is provided with openings 2 through which the secretion or other bodily fluids can be aspired. The suction tube 1 is terminated at its end which is to be inserted into the body by a material depot 3, illustrated in a side view. The suction tube 1 is connected to a low pressure location (not illustrated), so that the secretion is aspired in the direction of arrow A. The suction tube 1 is plugged at its end arranged in the body by the shaft 4 of the material depot 3. The region of the material depot 3 extending out of the shaft 4 is in the shape of a roller. The surface of the material depot 3 is enlarged by grooves 5 arranged along the suction tube axis. The material depot 3 contains the antibacterial active ingredient material (not illustrated). The material with the antibacterial action leaves the material depot 3 until it is aspired through the openings 2.

The embodiment illustrated in FIG. 2 corresponds to the one illustrated in FIG. 1, the difference being that the region extending out of the shaft 4 of the material depot 3 has a teardrop shape. The teardrop shape tapering of the material depot 3 affords the possibility of a simpler removal of the aspiration drainage system.

The embodiment illustrated in FIG. 3 corresponds to the one illustrated in FIG. 1, the difference being that the material depot 3 plugged onto the suction tube 1 was lengthened by two further material depots 3 with a spherical shape, which are fixed on a central wire 6.

The embodiment illustrated in FIG. 4 is similar to the one illustrated in FIG. 3. A chain of roller-shaped material depots 3 is fixed to a central wire 6 and a terminal material depot chain element 3 is plugged to the suction tube 1. The roller-shaped material depots 3 have concave-convex surfaces which correspond to each other in place of the opposing planar surfaces usual in the case of a cylinder. As a result of this, an articulated movement of the chain elements is possible, even in the case of a small distance between the adjacent chain elements 3.

1. An aspiration drainage system for aspirating secretions and other fluids out of a human or animal body using a suction tube and at least one material depot, arranged at an end of the suction tube to be inserted into the body, for dispensing an antibacterial active ingredient material, wherein a surface of the material depot is larger than an envelope of the material depot.

2. The aspiration drainage system of claim 1, wherein the surface of the material depot is larger than the envelope of the material depot by at least a factor of 1.1.

3. The aspiration drainage system of claim 1 or 2, wherein the material depot has at least one surface structure selected from the group composed of: depressions, hollow recesses, indentations and protuberances such as channels, folds, slits, lamellae, combs and bores.

4. The aspiration drainage system of claim 3, wherein channels, folds and slits are established along, across or, in a thread-like manner, in the surface of the material depot.

5. The aspiration drainage system of claim 1, wherein the antibacterial active ingredient material is enclosed in at least one of a carrier material intended to be reabsorbed by the body and a carrier material not intended to be reabsorbed by the body.

6. The aspiration drainage system of claim 1, wherein the material thickness between adjacent structures of the material depot does not exceed 5 mm.

7. The aspiration drainage system of claim 1, wherein the material depot has, at least in parts, open pores on its surface.

8. The aspiration drainage system of claim 1, wherein the material depot is shaped as a cylinder, sphere, cone, or combination of these basic shapes such as a teardrop shape.

9. The aspiration drainage system of claim 1, wherein the material depot is arranged detachably at the end of the suction tube to be inserted into the body.

10. A aspiration drainage system comprising at least two material depots according to claim 1 as elements in a chain.

11. The aspiration drainage system of claim 10, wherein opposing faces of adjacent material depot chain elements have a corresponding shape, at least in part.

12. The aspiration drainage system of claim 11, wherein the opposing faces of the adjacent material depot chain elements have a concave-convex design.

13. The aspiration drainage system of claim 2, wherein the surface of the material depot is larger than the envelope of the material depot by at least a factor of 2.

14. The aspiration drainage system of claim 13, wherein the surface of the material depot is larger than the envelope of the material depot by at least a factor of 3.

15. The aspiration drainage system of claim 4, wherein the channels, folds and slits are established along, across or, in a thread-like manner, in the surface of the material depot along an axis of the aspiration drainage tube or in a thread-like manner about the axis of the aspiration drainage tube.

16. The aspiration drainage system of claim 6, wherein the material thickness between the adjacent structures of the material depot does not exceed 3 mm.

17. The aspiration drainage system of claim 16, wherein the material thickness between the adjacent structures is between 1 and 3 mm.
